(19) United States
(12) Patent Application Publication
(10) Pub. No.: US 2008/0167488 A1
(43) Pub. Date: Jul. 10, 2008

(54) Process for the Preparation of Geminal ((Difluorocycloalkyl)Methyl) Amines

(75) Inventor: Maxime Tremblay, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/996,509

(22) PCT Filed: Jul. 26, 2006
(86) PCT No.: PCT/SE06/00910

§ 371 Date: Jan. 23, 2008

(30) Foreign Application Priority Data
Jul. 29, 2005 (SE) 0501756-1

(51) Int. Cl. C07C 209/48; C07C 253/30

(52) U.S. Cl. 558/431; 564/448

(57) ABSTRACT

The present invention relates to a process of preparing a compound of formula II, wherein R¹, n, m and p are defined in the specification.

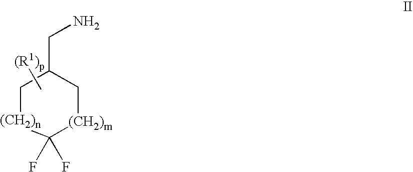

### FIELD OF INVENTION

The present invention relates to a process of making a difluoro compound, and particularly to a process of making a difluoro compound containing an amino group.

### BACKGROUND

Difluoro compounds containing an amino group are useful intermediates in the synthesis of compounds having therapeutic effects. PCT publication No. WO2004/108688 describes a method of making one of these difluoro compounds containing an amino group, [(4,4-difluorocyclohexyl)methyl]amine. However, an improved process of making these compounds is still desirable.

### DESCRIPTION OF THE EMBODIMENTS

In one aspect, the present invention provides a process of making a compound comprising:

a first step of reacting a compound of formula I with (dialkylamino)sulfur trifluoride to form a first product,

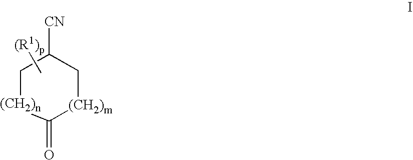

wherein R¹ is selected from hydrogen, C₁₋₆alkyl, C₂₋₆alkenyl, halogenated C₁₋₆alkyl, C₁₋₆alkoxy, —OH, and amino; and

n, m, and p are independently selected from 0, 1 and 2.

As used here, the term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloallyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amino” refers to —NH₂.

Halogen includes fluorine, chlorine, bromine and iodine.

“Halogenated,” used as a prefix of a group, means one or more hydrogens on the group is replaced with one or more halogens.

“RT” or “rt” means room temperature.

The term “Ra—Ni” refers to Raney® nickel such as Raney® nickel.

In one embodiment, the (dialkylamino)sulfur trifluoride used in the process is selected from (dimethylamino)sulfur trifluoride, (diethylamino)sulfur trifluoride and (morpholino)sulfur trifluoride.

In another embodiment, R¹ is selected from hydrogen and C₁₋₆alkyl.

In a further embodiment, n, m and p are 1 and R¹ is hydrogen.

In another embodiment, in the process of making a compound, the compound of formula I and (dialkylamino) sulfur trifluoride are reacted at a mole ratio between 1:10 and 10:1. Particularly, the mole ratio between the compound of formula I and (dialkylamino)sulfur trifluoride are between 2:1 and 1:2. More particularly, the mole ratio is about 1:1.1.

In another embodiment, the first step of reacting the compound of formula I and (dialkylamino)sulfur trifluoride is carried out at a temperature between −25° C. and 150° C. Particularly, the reaction temperature is between −25° C. and 25° C. More particularly, the reaction temperature is about 0° C.

In another embodiment, the process of making a compound further includes a purification step of extracting the first compound with water and a water-insoluble solvent from the reaction mixture. Particularly, the water-insoluble solvent is selected from chloroform, dichloromethane, and dichloroethane. More particularly, the water-insoluble solvent is dichloromethane.

In a further embodiment, the process of making a compound further includes another purification step of purifying the first compound by chromatography such as flash column chromatography using a suitable eluent.

In an even further embodiment, the process of making the compound further comprises a second step of reacting said first compound with a reducing agent to form a compound of formula II

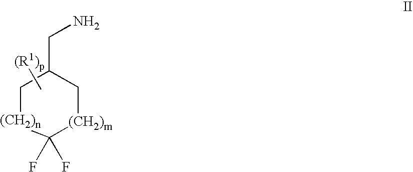

wherein R¹ is selected from hydrogen, C₁₋₆alkyl, C₂₋₆alkenyl, halogenated C₁₋₆alkyl, C₁₋₆alkoxy, —OH, and amino; and

n, m, and p are independently selected from 0, 1 and 2.

In one embodiment, the reducing agent is selected from a sodium or lithium aluminum hydride, sodium borohydride, potassium borohydride, lithium trimethoxy borohydride, lithium cyanoborohydride, sodium triacetoxyborohydride, potassium hydride, calcium hydride, sodium hydride, and hydrogen with Ra—Ni. Particularly, the reducing agent is hydrogen with Ra—Ni and lithium aluminum hydride.

In one embodiment, R¹ of formula II is selected from hydrogen and C₁₋₆alkyl.

In another embodiment, n, m and p of formula II are 1 and R¹ is hydrogen.

In another embodiment, the second step of the reaction is carried out at a temperature between 0° C. and 150° C. in a polar solvent such as tetrahydrofuran. Particularly, the reaction temperature is between 50° C. and 100° C. More particularly, the reaction temperature is about 75° C.

In another embodiment, the compound of formula II may be further purified using common purification procedures for organic compounds, such as distillation, extraction, and chromatography.

In another embodiment, the compound of formula I may be prepared by a process of reacting a compound of formula III with water and a hydrolysis catalyst,

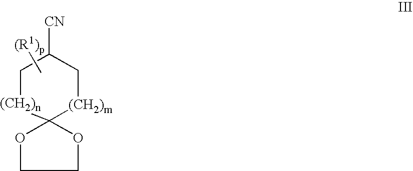

wherein R¹ is selected from hydrogen, C₁₋₆alkyl, C₂₋₆alkenyl, halogenated C₁₋₆alkyl, C₁₋₆alkoxy, —OH, and amino; and

n, m, and p are independently selected from 0, 1 and 2.

In a particular embodiment, R¹ of formula III is selected from hydrogen and C₁₋₆alkyl. In a more particular embodiment, n, m and p of formula III are 1 and R¹ is hydrogen.

In one embodiment, the ethylene acetal or ketal (1,3-dioxolane derivative) of formula III may be replaced with other type of ketone protecting group such as those illustrated in T. W. Greene & P. G. M. Wuts, Protective Groups in Organic Synthesis (2nd edition) J. Wiley & Sons, 1991.

In one embodiment, the hydrolysis catalyst is an acid. Particularly, the hydrolysis catalyst is ammonium cerium (IV) nitrate.

In another embodiment, the step of reacting a compound of formula III with water may be carried out at a temperature between 0° C. and 100° C. Particularly, the reaction is carried out at about 70° C.

In one embodiment, the compound of formula III may be prepared by a process including:

reacting a compound of formula IV with a tosylmethyl isocyanide at a temperature between −25° C. and 150° C.,

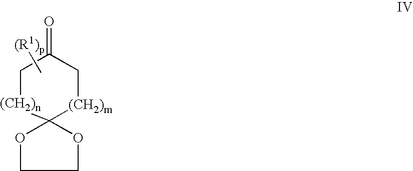

wherein R¹ is selected from hydrogen, C₁₋₆alkyl, C₂₋₆alkenyl, halogenated C₁₋₆alkyl, C₁₋₆alkoxy, —OH, and amino; and

n, m, and p are independently selected from 0, 1 and 2.

In a particular embodiment, R¹ of formula IV is selected from hydrogen and C₁₋₆alkyl. In a more particular embodiment, n, am and p of formula IV are 1 and R¹ is hydrogen.

In a further embodiment, the present invention provides a process of making a compound of formula II including the steps of:

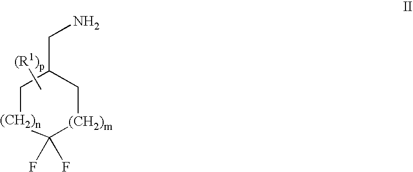

a) reacting a compound of formula IV with a tosylmethyl isocyanide at a temperature between −25° C. and 150° C. to form a compound of formula III,

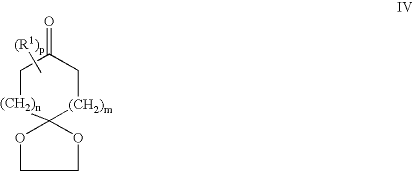

b) reacting a compound of formula III with water and a hydrolysis catalyst to form a compound of formula I,

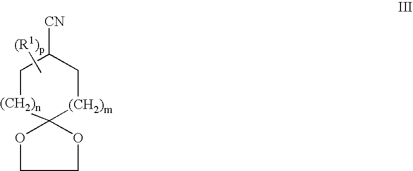

c) reacting a compound of formula I with (dialkylamino)sulfur trifluoride to form a compound of formula V,

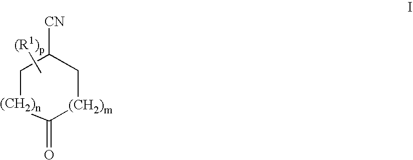

d) reacting a compound of formula V with a with a reducing agent to form the compound of formula II,

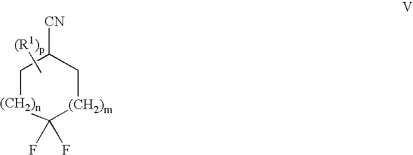

wherein R¹ is selected from hydrogen, C₁₋₆alkyl, C₂₋₆alkenyl, halogenated C₁₋₆alkyl, C₁₋₆alkoxy, —OH, and amino; and

n, m, and p are independently selected from 0, 1 and 2.

In a particular embodiment, R¹ is selected from hydrogen and C₁₋₆alkyl. In a more particular embodiment, n, am and p are 1 and R¹ is hydrogen.

### EXAMPLES

### 1,4-Dioxaspiro[4.5]decane-8-carbonitrile

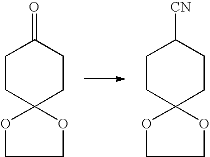

A solution of t-BuOK (22.8 g, 0.203 mol) in a 1:1 mixture of t-BuOH and 1,2-dimethoxyethane (200 mL) was added to a solution of 1,4-cyclohexanedione monoethylene ketal (15.5 g, 0.099 mol) and tosylmethyl isocyanide (20.3 g, 0.104 mol) in dimethoxyethane (200 mL) at 0° C. The reaction mixture was stirred for one hour at 0° C., allowed to warn to ambient temperature and stirred for one extra hour. The reaction mixture was poured in water (500 mL). The product was extracted with hexane (3×200 mL) and ether (3×200 mL). The combined organics were dried with anhydrous Na₂SO₄ and the solvent was concentrated. The product was purified by flash chromatography on silica gel using EtOAc 40% in hexane as eluent to afford the title compound as a colorless liquid. Yield: 11.5 g (69%). ¹H NMR (400 MHz, METHANOL-D₄): δ 1.55-1.69 (m, 2 H), 1.70-1.80 (m, 2H), 1.78-1.90 (m, 2H), 1.90-2.05 (m, 2H), 2.71-2.89 (m, 1H), 3.86-3.98 (m, 4H).

### 4-Oxocyclohexanecarbonitrile

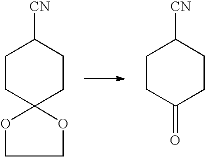

A solution of ammonium cerium (IV) nitrate (3.70 g, 6.75 mmol) in water (100 mL) was added to a solution of 1,4-dioxaspiro[4.5]decane-8-carbonitrile (11.3 g, 67.5 mmol) in MeCN (200 mL) and water (100 mL) at 70° C. The reaction mixture was stirred for 45 minutes at 70° C. and cooled to ambient temperature where 100 mL of water were added. The product was extracted with ether (4×200 mL). The combined organics were washed with brine and dried over MgSO₄. The product was purified by flash chromatography on silica gel using EtOAc 50% in hexane as eluent to afford the title compound as a colorless liquid. Yield: 6.30 g (75%). ¹H NMR (400 MHz, CHLOROFORM-D): δ 2.07-2.31 (m, 4H), 2.34-2.53 (m, 2H), 2.54-2.72 (m, 2H), 2.95-3.14 (m, 1H).

### 4,4-Difluorocyclohexanecarbonitrile

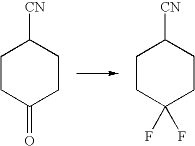

A solution of (Diethylamino)sulfur trifluoride (46.7 g, 289 mmol) in DCM (200 mL) was slowly added to a solution of 4-oxocyclohexanecarbonitrile (32.4 g, 263 mmol) in DCM (400 mL) at 0° C. The reaction mixture was stirred for 2 hrs. and quenched with water (100 mL). The organic layer was separated and dried with anhydrous MgSO₄. The solvent was concentrated to provide a 7:3 mixture of 4,4-Difluorocyclohexanecarbonitrile and 4-fluorocyclohex-3-ene-1-carbonitrile. MCPBA 77% (45 g, 200 mmol) was stirred in CHCl₃ (600 mL) for 30 min. The solution was dried with anhydrous Na₂SO₄ and filtered. The 7:3 mixture of 4,4-Difluorocyclohexanecarbonitrile and 4-fluorocyclohex-3-ene-1-carbonitrile was added to the resulting MCPBA solution and stirred at ambient temperature for 18 hrs. The reaction mixture was washed with NaOH 1.5 M (3×300 mL) and dried with anhydrous MgSO₄. The solvent was concentrated and the residue was distilled under reduced pressure to provide the pure title compound as a colorless liquid. Yield 18.2 g (47%): ¹H NMR (400 MHz, CHLOROFORM) δ 1.86-1.96 (m, 1H), 1.95-2.03 (m, 5H), 2.03-2.22 (m, 2H), 2.68-2.85 (m, 1H).

### [(4,4-Difluorocyclohexyl)methyl]amine

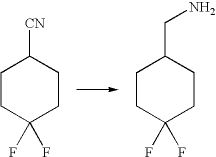

LiAlH₄ (2.20 g, 57.9 mmol) was added to a solution of 4,4-difluorocyclohexanecarbonitrile (6.10 g, 42.0 mmol) in THF (250 mL) at ambient temperature. The reaction mixture was refluxed to 75° C. and stirred for four hours. The reaction mixture was cooled to 0° C. and slowly quench by addition of MeOH over 1 hour. The mixture was filtered over a Celite pad and the filtrate was concentrated. The product was purified by distillation (65° C./15 mmHg) to afford the title compound as a colorless liquid. Yield: 2.55 g (40%). ¹H NMR (400 MHz, CHLOROFORM-D): δ 1.17-1.46 (m, 3H), 1.52-1.94 (m, 2H), 2.03-2.32 (m, 2H), 2.57-2.70 (m, 2H).

### [(4,4-Difluorocyclohexyl)methyl]amine

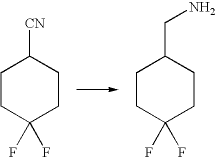

Raney® 2800 nickel, slurry in water, active catalyst (6 mL) was added to a solution of 4,4-difluorocyclohexanecarbonitrile (18.2 g, 125 mmol) in EtOH (300 mL). The reaction mixture was shaken overnight in a Parr® hydrogenation apparatus under a 50 PSI atmosphere of hydrogen. The reaction mixture was filtered over a celite pad and the solvent was concentrated to provide the title compound as a colorless liquid. Yield 16 g (87%). ¹H NMR (400 MHz, CHLOROFORM-D): δ 1.17-1.46 (m, 3H), 1.52-1.94 (m, 2H), 2.03-2.32 (m, 2H), 2.57-2.70 (m, 2H).

What is claimed is:

1. A process of making a compound comprising:
a first step of reacting a compound of formula I with (dialkylamino)sulfur trifluoride to form a first compound, 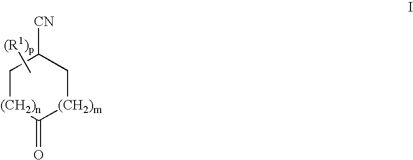
wherein R¹ is selected from hydrogen, C₁₋₆alkyl, C₂₋₆alkenyl, halogenated C₁₋₆alkyl, C₁₋₆alkoxy, —OH, and amino; and
n, m, and p are independently selected from 0, 1 and 2.

2. A process as claimed in claim 1, wherein said (dialkylamino)sulfur trifluoride is selected from (dimethylamino)sulfur trifluoride, (diethylamino)sulfur trifluoride and (morpholino)sulfur trifluoride.

3. A process as claimed in claim 1, wherein said process further comprises:
a second step of reacting said first compound with a reducing agent to form a compound of formula II 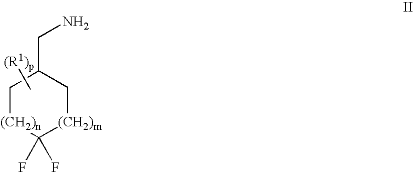
wherein R¹ is selected from hydrogen, C₁₋₆alkyl, C₂₋₆alkenyl, halogenated C₁₋₆alkyl, C₁₋₆alkoxy, —OH, and amino; and
n, m, and p are independently selected from 0, 1 and 2.

4. A process as claimed in claim 3, wherein said reducing agent is selected from a sodium or lithium aluminum hydride, sodium borohydride, potassium borohydride, lithium trimethoxy borohydride, lithium cyanoborohydride, sodium triacetoxyborohydride, potassium hydride, calcium hydride, hydrogen with Ra—Ni, and sodium hydride.

5. A process as claimed in claim 3, wherein said reducing agent is selected from hydrogen with Ra—Ni and lithium aluminum hydride.

6. A process as claimed in claim 1, wherein said compound of formula I and said (dialkylamino)sulfur trifluoride are reacted at a mole ratio between 1:10 and 10:1.

7. A process as claimed in claim 1, wherein said first step of reacting is carried out at a temperature between −25° C. and 150° C.

8. A process as claimed in claim 1, wherein said process further comprises a purification step of extracting said first product with water and a water-insoluble solvent.

9. A process as claimed in claim 1, wherein said process further comprises a third step of reacting a compound of formula III with water and a hydrolysis catalyst, 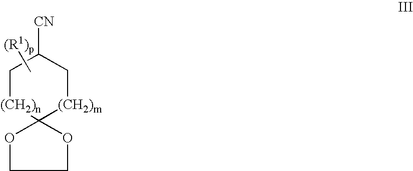
wherein R¹ is selected from hydrogen, C₁₋₆alkyl, C₂₋₆alkenyl, halogenated C₁₋₆alkyl, C₁₋₆alkoxy, —OH, and amino; and
n, m, and p are independently selected from 0, 1 and 2.

10. A process as claimed in claim 9, wherein said hydrolysis catalyst is ammonium cerium (IV) nitrate.

11. A process as claimed in claim 9, wherein said third step of reacting is carried out at a temperature between 0° C. and 150° C.

12. A process as claimed in claim 9, wherein said process further comprises a fourth step of reacting a compound of formula IV with a tosylmethyl isocyanide at a temperature between −25° C. and 150° C., 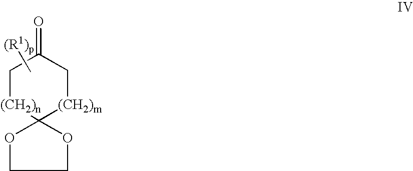
wherein R¹ is selected from hydrogen, C₁₋₆alkyl, C₂₋₆alkenyl, halogenated C₁₋₆alkyl, C₁₋₆alkoxy, —OH, and amino; and
n, m, and p are independently selected from 0, 1 and 2.

13. A process of making a compound comprising reacting a compound of formula V with a with a reducing agent, 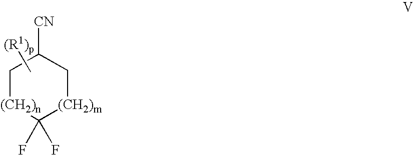
wherein R¹ is selected from hydrogen, C₁₋₆alkyl, C₂₋₆alkenyl, halogenated C₁₋₆alkyl, C₁₋₆alkoxy, —OH, and amino; and
n, m, and p are independently selected from 0, 1 and 2.

14. A process as claimed in claim 13, wherein said reducing agent is selected from a sodium or lithium aluminum hydride, sodium borohydride, potassium borohydride, lithium trimethoxy borohydride, lithium cyanoborohydride, sodium triacetoxyborohydride, potassium hydride, calcium hydride, hydrogen with Ra—Ni, and sodium hydride.

15. A process as claimed in claim 13, wherein said reducing agent is selected from hydrogen with Ra—Ni and lithium aluminum hydride.

16. A process as claimed in claim 13, wherein said reacting is carried out at a temperature between 0° C. and 150° C. for at least half an hour.

17. A process as claimed in claim 13, wherein said reacting is carried out in a polar solvent.

18. A process of making a compound of formula II including the steps of: 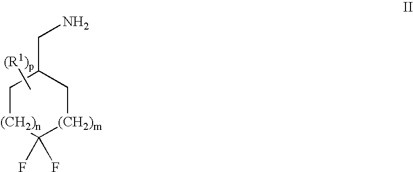
a) reacting a compound of formula IV with a tosylmethyl isocyanide at a temperature between −25° C. and 150° C. to form a compound of formula III, 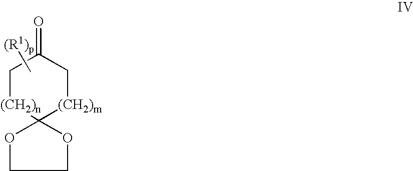
b) reacting a compound of formula III with water and a hydrolysis catalyst to form a compound of formula I, 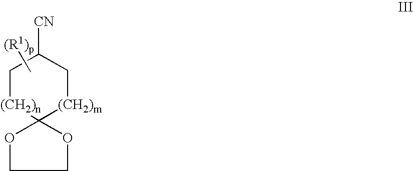
c) reacting a compound of formula I with (dialkylamino)sulfur trifluoride to form a compound of formula V, 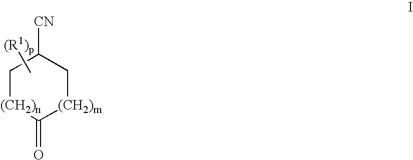
d) reacting a compound of formula V with a with a reducing agent to form the compound of formula II, 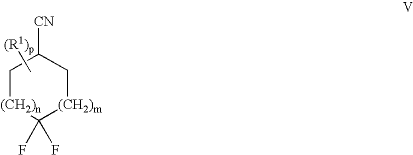
wherein R¹ is selected from hydrogen, C₁₋₆alkyl, C₂₋₆alkenyl, halogenated C₁₋₆alkyl, C₁₋₆alkoxy, —OH, and amino; and
n, m, and p are independently selected from 0, 1 and 2.
